(19) United States
(12) Patent Application Publication
(10) Pub. No.: US 2025/0318998 A1
(43) Pub. Date: Oct. 16, 2025

(54) REMINERALIZING RESTORATIVE COMPOSITIONS AND METHODS THEREOF

(71) Applicant: ADA FORSYTH INSTITUTE, INC., Cambridge, MA (US)

(72) Inventors: Li ZHEN, Chengdu (CN); Jirun SUN, Rockville, MD (US); Felicitas BIDLACK, Somerville, MA (US)

(73) Assignee: ADA FORSYTH INSTITUTE, INC., Cambridge, MA (US)

(21) Appl. No.: 19/000,649

(22) Filed: Dec. 23, 2024

Related U.S. Application Data
(60) Provisional application No. 63/614,454, filed on Dec. 22, 2023.

(51) Int. Cl. A61K 6/884; A61K 6/838

(52) U.S. Cl. CPC A61K 6/884; A61K 6/838

(57) ABSTRACT

Compositions and methods of using the composition to repair dental material or treat a subject in need thereof.

### CROSS-REFERENCE TO RELATED APPLICATIONS

This application claims the benefit of U.S. Provisional Patent Application No. 63/614,454 filed Dec. 22, 2023, the contents of which is hereby incorporated by reference in its entirety.

### STATEMENT REGARDING FEDERALLY SPONSORED RESEARCH

This invention was made with government support under grant R21 DE029903 awarded by the National Institutes of Health. The government has certain rights in this invention.

### FIELD

This disclosure relates to restorative compositions, including, but not limited to, dental restorative compositions that can promote remineralization of hard dental tissues.

### BACKGROUND

Polymer-based composite materials may be used in a variety of applications, including, but not limited to, dental restorations and packaging. Over time, reactive oxygen species (ROS), thermal and/or mechanical stresses may cause material fatigue and micro-cracks. Mechanical stresses may arise because of strong occlusal forces, for example by chewing and clenching, thermal changes, and enzymes that digest the material. Each of these factors may cause cracking, abrasion, tension, and weakening of resin filler materials in dental applications. Without a repair mechanism, the micro-cracks may propagate and lead to corresponding catastrophic failure of the resin filler material. However, materials fatigue and micro-cracks are difficult to detect, and even if detected, may not be repairable in situ with currently available methods and materials. Instead, repair may require the complete removal and replacement of the resin filler material. Thus, there is a need for improved compositions and methods to mitigate, prevent, or treat ROS-induced or other material fatigue and micro-cracks.

### SUMMARY

In certain aspects, provided herein are compositions comprising, consisting essentially of, consisting of, a curable liquid; a filler; and an additive. In certain embodiments, the curable liquid may be a liquid component comprising one or more selected from the group consisting of a liquid component of a resin composite, a liquid component of a glass ionomer cement, a liquid component of a resin-modified glass ionomer cement, and a liquid component of a polyacid-modified resin composite. In certain embodiments, the liquid component may comprise one or more monomers in the resin composite. In certain embodiments, the one or more monomers may polymerize from a liquid state to a solid state. In certain embodiments, the liquid component may be a polymer solution in the glass ionomer cement. In certain embodiments, the polymer solution may be cured by chemical reaction. In certain embodiments, the liquid component may be a mixture of monomers and polymer solutions in the resin-modified glass ionomer cement or the polyacid-modified resin composite. In certain embodiments, the curable liquid may comprise about 10% to 99% of the composition by mass. In certain embodiments, the filler may comprise, consist essentially of, or consist of hydrogel particles and a filler. In certain embodiments, the filler may comprise, consist essentially of, or consist of hydrogel particles and calcium phosphate particles. In certain embodiments, the hydrogel particles may be made by natural materials extracted from animal products and plants. In certain embodiments, the natural materials may be selected from one or more materials from the group consisting of silk fibroin (SF), tannic acid, chitosan, alginate, collagen, and gelatin. In certain embodiments, the hydrogel particles may be made by synthetic materials. In certain embodiments, the hydrogel particles may be selected from one or more of the group consisting of hyaluronic acid, polyvinyl alcohol, polyacrylamide, poly (N-isopropylacrylamide) (PNIPAAm), poly (acrylic acid) (PAA), polyvinylpyrrolidone (PVP), poly (ethyleneimine) (PEI) polyurethane hydrogel particles, and poly(2-hydroxyethyl methacrylate) (PHEMA). In certain embodiments, the hydrogel particles may comprise about 0.1% to 30% by mass of the composition. In certain embodiments, the size of the hydrogel particles may be within the range of 20 nm to 100 μm. In certain embodiments, the composition may further comprise a healing agent. In certain embodiments, the healing agent may be one or more selected from the group consisting of a peptide, a bisphosphonate, and a bisphosphonate derivative. In certain embodiments, the healing agent may be encapsulated in the hydrogel particles, attached on the surface of the hydrogel particles, or a combination thereof. In certain embodiments, the healing agent may comprise about 0.01-15% of the hydrogel particles composition by mass. In certain embodiments, the healing agent may guide the calcium phosphate production onto or next to hydrogel particles containing the healing agent compared to random calcium phosphate production in a composition that does not include the healing agent. In certain embodiments, the healing agent may attach onto tooth and bone. In certain embodiments, the peptide may be one or more selected from the group consisting of a polyaspartic acid peptide, an aspartate-serine-serine (DSS) peptide, and an acidic peptide. In certain embodiments, the healing agent may be dispersed in curable liquid directly without hydrogel particles. In certain embodiments, the composition may comprise 0.1% to 30% of calcium phosphate particles. In certain embodiments, the calcium phosphate particles may comprise one or more crystalline forms selected from the group consisting of amorphous calcium phosphate particles, hydroxy apatite, octacalcium phosphate, dicalcium phosphate (DCP), tricalcium phosphate (TCP), brushite, and monetite. In certain embodiments, the composition may further comprise one or more other fillers selected from the group consisting of prepolymerized fillers, silica, titanium dioxide, zirconium dioxide, fluoroaluminosilicate glass, calcium fluoride, fiber reinforcement, strontium-based glass, bioactive glass, aluminum oxide, barium oxide, yttrium oxide, magnesium oxide, calcium oxide, tin oxide, iron oxide, strontium oxide, and cerium oxide. In certain embodiments, the composition may comprise 0-75% of the other fillers. In certain embodiments, the additive may comprise one or more selected from the group consisting of an initiator, a co-initiator, an accelerator, an antimicrobial agent, a pigment, an opacifier, a stabilizer, and a coupling agent. In certain embodiments, the composition may be one or more selected from the group consisting of a dental restorative material, a dental filling material, a laminate veneer, a dental adhesive, a denture, an adhesive, a cement, and a sealant. In certain embodiments, the composition may be used for one or more selected from the group consisting of coating, high-end materials wrapping, and wound healing.

In certain aspects, provided herein are methods of repairing a dental material. In certain embodiments, the methods comprise contacting the dental material with any of the compositions disclosed herein.

In certain aspects, provided herein are methods of treating a subject having a tooth injury. In certain embodiments, the methods comprise contacting the tooth with any of the compositions disclosed herein.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model and are not necessarily to scale.

FIG. 1 shows the effect of the remineralization solution on carious lesions in the presence of 8DSS. In FIG. 1, human teeth were treated with buffer only (left column), remineralization solution alone (second column), remineralization solution with 10 μL 8DSS for 5 minutes (third column), or with remineralization solution with 10 μL 8DSS for 5 minutes (fourth column) and imaged using SEM. Backscatter electron micrographs (top row), cross sectional micrographs at 1000× (second row), cross sectional micrographs at 10,000× (third row), surface micrographs at 1000× (fourth row), and surface micrographs at 10,000× (fifth row) were taken for each treatment group. Scale bar: 10 μM at ×1000, and 1 ×M at ×10000.

FIG. 2 shows 7-day lesions on erupted permanent human molars treated with control or 5-carboxyfluorescein (5FAM)-8DSS. Human molars with intact enamel (left column) or enamel lesions (right column) were treated with buffer control (top row) or with 8DSS conjugated to 5FAM (“5FAM-8DSS”) (bottom row) and imaged with fluorescent imaging. ES: Enamel Surface. E: Enamel. NP: Nail polish covered surface. Scale bar: 100 μm.

FIGS. 3A-3C show a schematic of an example of a self-healing strategy leading to remineralization. In this example, the composite includes nanoparticles of amorphous calcium phosphate (NACP) and a tyramine substitution (TS) hydrogel containing 8DSS (“DSS”). As shown in FIG. 3A (top panel), a crack with simulated body fluid (SBF) may form in the composite. As shown in FIG. 3B (middle panel), the crack may pass into or through the DSS containing TS hydrogel, causing the release of DSS peptide into the crack. As shown in FIG. 3C (bottom panel), the release of DSS leads to remineralization of the micro-crack.

FIGS. 4A-4D show guided mineralization through the combination of amorphous calcium phosphate nanoparticles (NACPs) and 8DSS. FIG. 4A shows mineralization of silk-fibroin, tannic acid, and U/V resin. FIG. 4B shows mineralization of silk-fibroin, tannic acid, U/V resin with 8DSS. FIG. 4C shows mineralization of silk-fibroin, tannic acid, U/V resin with NACP. FIG. 4D shows mineralization of silk-fibroin, tannic acid, U/V resin with NACP and 8DSS.

FIG. 5 shows confocal microscopy images of the self-healing resin adhesive prepared in Example 2, appearing translucent in the upper portion of the imaged volume. The self-healing U/V resin (prepared in Example 2) contains both nanoparticles of amorphous calcium phosphate and Texas Red X-labelled silk-fibroin and tannic acid hydrogel particles (light grey spheres) with 5FAM-8DSS overlaying dentin, which is opaque and visible by autofluorescence in the lower portion of the volume imaged by confocal microscopy. The hydrogel spheres are below 3 micro-meter in diameter and evenly distributed adjacent to dentin. Scale bar: 10 microns.

FIG. 6 shows scanning electron microscopy images that illustrate the infiltration of resin into dentin tubules and the formation of resin tags that is equal between the experimental self-healing resin (the silk-fibroin, tannic acid, 8DSS, NACP, and U/V composition as prepared in Example 2) (two left columns, Experimental A and Experimental B) and the multipurpose Scotchbond adhesive, used as control (two right columns, Control A and Control B). Scale bar in all images: 1 micron.

### DETAILED DESCRIPTION

### Definitions

As used herein, the word “about” when immediately preceding a numerical value means a range of plus or minus 10% of that value, e.g., “about 50” means 45 to 55, “about 10” means “9 to 11,” etc. Furthermore, the phrases “less than about” a value or “greater than about” a value should be understood in view of the definition of the term “about” provided herein.

Where ranges are provided herein, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

### Compositions

Provided herein are compositions that may be used for restorative dentistry, specifically dental composite materials that can be used with or without other fillers as restorative materials, filling materials, laminate veneers, adhesives, denture, dental adhesives, cement, sealant, materials for additive manufacturing, and more. Additional applications may include the generation of interfaces between different types of mineralized structures, such as dentin and enamel, or mineralized tissues such as dentin, cementum, and bone. In addition to applications in dentistry and mineralized tissue treatments, the compositions disclosed herein may be useful as adhesive and repair material in aqueous environments, and for coating, and high-end materials wrapping. For example, in certain embodiments, the compositions may be used for coating or laminating as insulative and protective wrapping in electrical applications, advanced composite wrapping in aerospace and automotive, or wrapping and coating components exposed to saltwater and UV radiation, such as boat hulls, masts, and underwater structures. In certain embodiments, the compositions disclosed herein may be used for wound healing.

The compositions, the methods to prepare them, and the methods to apply them can prevent, or stop propagation of cracks, ruptures, and degradation; increase resistance to reactive oxygen species (ROS); and restore the function of the materials through one or more of the effects of hydrogen-bonding (H-bonding), mineralization, and chemical interaction.

In certain embodiments, the compositions provided herein can be added to the surface of a material to enhance mineralization and strength of the material. The material may be a dental material, enamel, dentin, bone, or any other surface. For example, the composition may be a glass ionomer cement. The composition may comprise a polymer solution, e.g., aqueous solution of polyacrylic acid or a similar polyacid, in a liquid state, an acid-reactive fluoroaluminosilicate glass, a calcium phosphate particle, and a healing agent encapsulated in a hydrogel particle, wherein the polyacid reacts with the acid-reactive fluoroaluminosilicate glass to form a solid state. The composition may further comprise another filler and/or an additive.

In certain embodiments, the compositions herein may comprise a resin composite, a hydrogel particle, and a healing agent. One aspect of the disclosure provides a dental restorative composition that comprises a curable liquid comprising polymerizable monomers, fillers, and additives. In certain embodiments, the monomer may be cured or polymerized from a liquid state to a solid state. In certain embodiments, the fillers may comprise hydrogel particles, calcium phosphate particles, with/without other particles. In certain embodiments, the healing agent may comprise a peptide or compounds that promote or guide calcium phosphate formation. In certain embodiments, the healing agent may be encapsulated in the hydrogel particle or attached on the surface of the hydrogel particle. In certain embodiments, the healing agent may induce nuclei of calcium phosphate particles. In certain embodiments, the peptide may comprise tripeptide aspartate-serine-serine (DSS) or its derivatives. In certain embodiments, the additives may comprise initiator systems to initiate curing of monomers via light, heat or both.

In certain embodiments, the compositions disclosed herein may comprise a curable liquid as disclosed herein, fillers as disclosed herein, and additives as disclosed herein. In certain embodiments, the composition may be, without limitation, a dental restorative material, dental filling material, laminate veneer, dental adhesive, denture, adhesive, cement, or sealant. In certain embodiments, the composition may be used for as adhesive and repair material in aqueous environments, coating, high-end materials wrapping, and/or wound healing.

### Curable Liquid

In certain embodiments, the compositions disclosed herein may comprise a curable liquid. In certain embodiments, the curable liquid may be the liquid component of a resin composite, the liquid component of a glass ionomer cement, the liquid component of a resin-modified glass ionomer cement, or the liquid component of a polyacid-modified resin composite.

In certain embodiments, the liquid component may be a monomer or mixture of monomers in resin composites. In certain embodiments, the monomer or mixture of monomers in resin composites may be polymerized from a liquid state to a solid state.

In certain embodiments, the liquid component may be a polymer solution in a glass ionomer cement. In certain embodiments, the polymer solution may be cured by chemical reaction.

In certain embodiments, the liquid component may be a mixture of monomers and/or polymer solutions in a resin-modified glass ionomer cement or a polyacid-modified resin composite.

In certain embodiments, the curable liquid may include monomers, polymers, or resins that are liquids and can form a solid crosslinked resin network upon polymerization,

In certain embodiments, the curable liquid may comprise about 10% to 99% of the composition by mass. In some embodiments, the curable liquid may comprise about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% by mass of the composition. In some embodiments, the curable liquid may comprise 10%, 15%, 20%, 25%, 30%, 35%, 40%, 45%, 50%, 55%, 60%, 65%, 70%, 75%, 80%, 85%, 90%, 95% or 99% by mass of the composition. In some embodiments, the curable liquid may comprise about 10-99%, about 60-90%, or about 75-85% of the composition by mass of the composition. In some embodiments, the curable liquid may comprise about 80% of the composition by mass of the composition.

### Monomers

In certain embodiments, the curable liquid disclosed herein may comprise a monomer. As used herein, a “monomer” binds or cross links with another monomer to form a polymer. Combinations of monomers may also be used. Examples of polymerizable monomers are described in U.S. Pat. No. 10,246,540 B2, which is incorporated in its entirety herein.

In certain embodiments, the monomer may polymerize and transform or cure from a liquid state to a solid state. In some embodiments, the monomer may comprise one or more monomers. The one or more monomers may include, without limitation, vinyl-containing monomer, including methacrylate derivative, styrene derivative, and acrylate-derivative; for example, urethane dimethacrylate (UDMA), triethylene glycol divinylbenzyl ether (TEGDVBE), exothane 9, E6, Bis-GMA, and triethylene glycol dimethacrylate (TEGDMA); cyclic ester, cyclic ether, cyclic siloxane, cyclic amide, and cyclic carbonate. For example, the monomer may include UDMA and TEGDVBE. In certain embodiments, the monomer may include Bis-GMA and TEGDMA. In certain embodiments, the composition may comprise more than one monomer in equal parts (i.e., in a 1:1 ratio). For example, the composition may comprise the monomers UDMA and TEGDVBE at a ratio of 1:1. In some embodiments, the composition may comprise more than one monomer at various ratios (i.e., two monomers at a ratio of 6:4, 7:3, 8:2, or 9:1). For example, the composition may comprise the monomers Bis-GMA and TEGDMA at a ratio of 7:3.

### Solution of Polyacid

In certain embodiments, the curable liquid disclosed herein may comprise a solution of polyacids. In certain embodiments, the solution of polyacids may be the liquid phase of a glass ionomer cement (GIC), which is responsible for the acid-base reaction with the fluoroaluminosilicate glass powder. In certain embodiments, the solution of polyacids contributes to the setting process, bonding, and mechanical properties of GIC.

In certain embodiments, the curable liquid disclosed herein may comprise an aqueous solution of polyacids that comprise one or more acidic polymers or acidic compounds for GIC. The acidic polymers or acidic compounds may be selected from the group consisting of polyacrylic acid, itaconic acid, maleic acid, tartaric acid, poly (maleic-co-acrylic) acid, and citric acid.

### Fillers

In certain embodiments, the compositions disclosed herein may include a filler. In certain embodiments, the filler may include one or more fillers. As used herein, a “filler” includes one or more compounds that provide structure to the composition. In certain embodiments, the filler may be for mineralization and reinforcement of the composition when it is in its solid state.

In certain embodiments, the filler may comprise hydrogel particles and one or more other fillers. For example, in certain embodiments, the filler may comprise hydrogel particles and calcium phosphate particles. In certain embodiments, the composition may comprise only hydrogel particles and calcium phosphate particles as fillers. In certain embodiments, the composition may comprise hydrogel particles, calcium phosphate particles, and one or more other fillers. In certain embodiments, the filler may be calcium phosphate particles and/or metal oxide particles. In some embodiments, the calcium phosphate particles may be any crystalline form including amorphous calcium phosphate particles, hydroxy apatite, octacalcium phosphate, dicalcium phosphate (DCP), tricalcium phosphate (TCP), brushite, and monetite. In some embodiments, the composition comprises about 0.1%, 1%, 2%, 3%, 4%, 5%, 6%, 7%, 8%, 9%, 10%, 12%, 15%, 17%, 20%, 25%, or 30% of calcium phosphate particles by mass of the composition. In some embodiments, the composition comprises about 0.1-30%, about 5-20%, about 5-15%, about 8-12%, or about 10%, of the calcium phosphate particles by mass of the composition. In some embodiments, the composition comprises 0.1-30%, 5-20%, 5-15%, 8-12%, or 10%, of the calcium phosphate particles by mass of the composition. In some embodiments, the composition includes amorphous calcium phosphate nanoparticles (NACPs).

In certain embodiments, the composition comprises an amorphous calcium phosphate filler, the weight percentage of the composition is 10% amorphous calcium phosphate, 10% hydrogel-peptide using tannic acid silk hydrogel (TS) with 40 μM 8DSS peptide and SF-TA hydrogel, and 80% urethane dimethacrylate (UDMA)-triethylene glycol divinylbenzyl ether (TEGDVBE) (U/V) resin. The U/V resin is described, for example, in U.S. Pat. No. 10,246,540 B2, which is incorporated by reference in its entirety herein.

In certain embodiments, the composition may include one or more fillers other than hydrogel particles. For example, other fillers may include prepolymerized fillers, silica, titanium dioxide, zirconium dioxide, fluoroaluminosilicate glass, calcium fluoride, fiber reinforcement, strontium-based glass, bioactive glass, aluminum oxide, barium oxide, yttrium oxide, magnesium oxide, calcium oxide, tin oxide, iron oxide, strontium oxide and cerium oxide. In certain embodiments, the composition may comprise 0-75% of the other fillers.

In some embodiments, the filler can be dissolved by acid and remineralized around a hydrogel guided by a healing agent.

### Hydrogel Particles

In certain embodiments, the filler of the compositions disclosed herein may include hydrogel particles. The hydrogel particles used in the composition should be chemically compatible with the curable liquid components (e.g., monomers) with which they are mixed.

Hydrogel particles of the present disclosure may be made of natural products, synthetic materials, or a combination thereof. In certain embodiments, the hydrogel particles may be formed by natural materials extracted from animals and/or plants. For example, in certain embodiments, hydrogel particles may include, without limitation, silk fibroin (SF), tannic acid, chitosan, alginate, collagen, and/or gelatin. In certain embodiments, hydrogel particles may be made from synthetic materials. For example, in certain embodiments, hydrogel particles may include, without limitation, hyaluronic acid, polyvinyl alcohol, polyacrylamide, poly (N-isopropylacrylamide) (PNIPAAm), poly (acrylic acid) (PAA), polyvinylpyrrolidone (PVP), poly (ethyleneimine) (PEI), polyurethane hydrogels, and/or poly (2-hydroxyethyl methacrylate) (PHEMA).

In some embodiments, the hydrogel particles comprise a natural or synthetic material. For example, chitosan with tannic acid, alginate with tannic acid, gelatin with tannic acid, hyaluronic acid with tannic acid, collagen with tannic acid, or polyvinyl alcohol (PVA) with tannic acid hydrogel particles. These examples illustrate the versatility of hydrogel particles that combine natural and/or synthetic materials, tailored for applications in biomedical and environmental fields.

In some embodiments, the composition may comprise more than one hydrogel particle in equal parts (i.e., in a 1:1 ratio). For example, the composition may comprise SF and TA at a ratio of 1:1. In some embodiments, the composition may comprise more than one hydrogel particle at various ratios (e.g., two hydrogel particles at a ratio of 6:4, 7:3, 8:2, or 9:1). For example, the composition may comprise SF and TA at a ratio of 7:3.

In some embodiments, the hydrogel particles may comprise about 0.1%, 1%, 2%, 3%, 4%, 5%, 6%, 7%, 8%, 9%, 10%, 12%, 15%, 17%, 20%, 25%, or 30% by mass of the composition. In some embodiments, the hydrogel particles may comprise about 1-30%, about 5-20%, about 5-15%, about 8-12%, or about 10% by mass of the composition. In some embodiments, the hydrogel particles may comprise 1-30%, 5-20%, 5-15%, 8-12%, or 10% by mass of the composition.

In certain embodiments, the size of the hydrogel particles may be within the range of 20 nm to 100 μm. In certain embodiments, the hydrogel particles may be nano-sized particles. In certain embodiments, the hydrogel particles may be micron-sized particles.

### Additives

In certain embodiments, the compositions disclosed herein may include an additive. As used herein, an “additive” includes compounds that provide an additional function to the composition. For example, in certain embodiments, the additive may provide the function of initiating polymerization or providing antimicrobial functions.

In certain embodiments, the additive may include one or more additives. In certain embodiments, the additive may be one or more selected from the group consisting of an initiator, a co-initiator, an accelerator, an antimicrobial agent, a pigment, an opacifier, a stabilizer, and a coupling agent.

In certain embodiments, the additive may be an initiator. In certain embodiments, the initiator may be a photoinitiator. In certain embodiments, photoinitiators may include a camphor quinone (CQ) and/or an amine, camphor quinone or derivatives, a combination of camphor quinone or derivatives and amine(s), including ethyl-4-N, N-dimethyl-aminobenzonate, or phenylpropanedione or derivatives, such as 1-phenyl-1,2-propanedione, or bisacrylphosphine oxide or derivatives including bis(2,4,6-trimethylbenzoyl)-phenylphosphineoxide (Irgacure 819), bis(2,6-dimethoxy benzoyl)-trimethylpentyl phosphine oxide & 1-hydroxycyclohexyl phenyl ketone, and with/without diaryl iodonium derivatives, and with/without boryl radicals including tert-butylamine borane complex.

Examples of initiators are described, for example, in U.S. Pat. No. 10,246,540 B2, which is incorporated by reference in its entirety herein. In some embodiments, the initiator is metal oxide. In some embodiments, the transition of the composition from liquid state to solid state is initiated without a chemical initiator, for example through heat or light without a chemical initiator.

In certain embodiments, the additives may comprise initiator systems to initiate curing of monomers via light, heat or both. In certain embodiments, the additives comprise initiator systems that accelerate polymerization with light irradiation or heat.

### Healing Agents

In certain embodiments, the compositions disclosed herein may include a healing agent. In certain embodiments, healing agents may be encapsulated in the hydrogel particles, attached on the surface of the hydrogel particles, or a combination thereof. In certain embodiments, the healing agents are not fixed in one location, they may diffuse out of the hydrogel over time. In some embodiments, the composition comprises a filler that comprises the healing agent. In some embodiments, the composition comprises an additive that comprises the healing agent. In certain embodiments, the healing agent may be dispersed in curable liquid directly without hydrogel particles.

Healing agents of the compositions disclosed herein may accelerate and/or guide/direct the formation of calcium phosphate or other metal oxide compared to a composition that does not include the healing agent. In some embodiments, the healing agent increases mineralization or remineralization compared to a composition that does not include the healing agent. For example, the healing agent may bind to a tooth surface and increase the precipitation of mineral crystals compared to a composition that does not include the healing agent. A tooth surface may include any exposed surface on the enamel, cementum, or dentin surface. In certain embodiments, the healing agent may guide the calcium phosphate production onto or next to hydrogel particles containing the healing agent compared to random calcium phosphate production in a composition that does not include the healing agent.

Healing agents of the compositions disclosed herein may include a peptide, a bisphosphonate, or bisphosphonate derivatives. In certain embodiments, the healing agent may attach onto mineralized tissues including tooth and bone. In some embodiments, the peptide includes a plurality of aspartic acid residues (i.e., a polyaspartic acid). In certain embodiments, the peptide may be a polyaspartic acid peptide, an aspartate-serine-serine (DSS) peptide, an acidic peptide, or a combination thereof. In some embodiments, the peptide comprises one or more amino acid sequence repeats that facilitate mineralization or remineralization. For example, the peptide may comprise one or more amino acid repeats of aspartate-serine-serine (DSS), asparagine-threonine-threonine (NTT), aspartate-threonine-threonine (DTT), glutamate-threonine-threonine (ETT), asparagine-serine-serine (NSS), glutamate-serine-serine (ESS), aspartate-alanine-alanine (DAA), alanine-serine-serine (ASS), or asparagine-alanine-alanine (NAA). In some embodiments, the peptide may include 2-12, 4-10, or 6-9 amino acid sequence repeats. In certain embodiments, the peptide includes 1, 2, 3, 4, 5, 6, 7, 8, 9, 10, 11, 12, 13, 14, or 15 amino acid sequence repeats. In some embodiments, the peptide comprises eight repeats of tripeptide aspartate-serine-serine (8DSS), or other repeats of tripeptide aspartate-serine-serine in phosphorylated or unphosphorylated form. Other peptides that may be used in the present disclosure include those that are known to facilitate calcium phosphate mineralization processes. Other examples of peptides that may be used are described by Yarborough 2010, which is incorporated by reference in its entirety herein.

In some embodiments, the bisphosphonate, or bisphosphonate derivatives may be selected from alendronate, ibandronate, zoledronic acid, risedronate, etidronate, pamidronate, tiludronate, zoledronic acid, and derivatives. In certain embodiments, these derivatives may attach onto mineralized tissues including tooth and bone.

In some embodiments, the hydrogel may comprise the healing agent. The concentration of the healing agent in the hydrogel may be about 5 μM, 10 μM, 15 μM, 20 HM, 25 μM, 30 μM, 35 μM, 40 μM, 45 μM, 50 μM, 55 μM, 60 μM, 65 μM, 70 M, 75 μM, 80 μM, 85 μM, 90 μM, 95 μM, 100 μM. In certain embodiments, the concentration of the healing agent in the hydrogel may be about 5-100 μM, about 20-80 μM, about 30-50 μM, or about 35-45 μM. In certain embodiments, the concentration of the healing agent in the hydrogel may be 5-100 μM, 20-80 μM, 30-50 M, or 35-45 μM. In some embodiments, the ratio of the peptide to hydrogel may be 0.6:1000-12:1000, for example 1.2:1000. In some embodiments, the composition comprises a healing agent without a hydrogel.

In certain embodiments, the healing agent may comprise about 0.01-15% by mass of the hydrogel particle composition. In certain embodiments, the healing agent may comprise about 0.01%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15% by mass of the hydrogel particle composition.

### Methods

The present disclosure also includes methods for repairing a dental material. In certain embodiments, the methods may include contacting the dental material with a composition of the present disclosure. As used herein, a “dental material” may include, for example, dentin, enamel, or any other material used to replace, repair, or replicate dentin and/or enamel, for example dental fillers or veneers. In a non-limiting example, a composition of the present disclosure may be used as a filler to replace a cracked or damaged tooth. In some embodiments, the composition may be used to repair a cavity. In some embodiments, the method of repairing dental material results in a repaired dental material that has increased longevity compared to a repaired dental material using standard methods (i.e., without a composition of the present disclosure). As used herein, the “longevity” of a repaired material refers to the period of time that passed before the repaired material needs to be replaced, for example due to damage or normal wear.

The present disclosure also includes methods of promoting anti-aging of dental material. Over time, reactive oxygen species (ROS), thermal and/or mechanical stresses may cause material fatigue and micro-cracks. The mechanical stresses may arise because of strong occlusal forces, for example by chewing and clenching, thermal changes, and enzymes that digest the material. Each of these factors may cause cracking, abrasion, tension, and weakening of resin filler materials in dental applications. Without a repair mechanism, the micro-cracks may propagate and lead to corresponding catastrophic failure of the resin filler material. However, materials fatigue and micro-cracks are difficult to detect, and even if detected, may not be repairable in situ with currently available methods and materials. Instead, absent self-healing materials, repair may require the complete removal and replacement of the resin filler material. The prevention of ROS induced material fatigue and the capacity to self-heal or self-repair micro-cracks hinges on the composition, homogeneity, and activity of the filler material. Therefore, the compositions and methods of the present disclosure may prevent or stop crack propagation and restore the function of the materials through H-bonding, mineralization, and chemical interaction.

The present disclosure also includes methods of treating a subject having a tooth injury comprising contacting the tooth with a composition of the present disclosure. As used herein, “treating” includes replacing, repairing, or replicating the dental material of the subject. Treating the subject may result in, for example, decreased pain, increased longevity of the dental material, or increased satisfaction of the subject compared to the subject before the treatment was administered, compared to a subject that did not receive the treatment, or compared to a subject that received a treatment different from the method of the present disclosure.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations of embodiments of the present disclosure. It is to be understood that the above description is intended to be illustrative, and not restrictive, and that the phraseology or terminology employed herein is for the purpose of description and not of limitation. Combinations of the above embodiments and other embodiments will be apparent to those of skill in the art upon studying the above description. The scope of the present disclosure includes any other applications in which embodiment of the above concepts and methods are used. The scope of the embodiments of the present disclosure should be determined with reference to claims associated with these embodiments, along with the full scope of equivalents to which such claims are entitled.

### EXAMPLES

The principles and embodiments described above are further illustrated by the non-limiting examples that follow:

### Example 1

### 8DDS Promotes Remineralization But Has Low Penetration Into Enamel

Micro-cracks are difficult to detect in part because the initial cracks propagate through the material and go unnoticed until breaking out of the filler or the entire tooth (e.g., oblique fractures). These internal cracks are almost impossible to repair manually without removing portions of the filler or tooth.

One solution to repairing micro-cracks is to create self-healing materials that repair the micro-cracks without external intervention. This would improve the longevity of the material and may even enhance performance including beneficial rigidity, e.g., improved hardness overtime, and functionality, e.g., antimicrobial and protein repellant. Such a material can be incorporated into dental composite materials that can be used as restorative materials, filling materials, laminate veneers, adhesives, dentures, dental adhesives, cements, sealants, materials for additive manufacturing, and more.

Current bioactive regenerative materials lack controls on where the remineralization takes place, for example current regenerative materials result in calcium release into oral environments rather than staying in the restoratives. In some embodiments, the present technology uses healing agents delivered using hydrogel to guide the formation of minerals. These healing agents may attach to dental substrate and mineralized tissues, including tooth and bone, and initiate calcium phosphate formation where they are located.

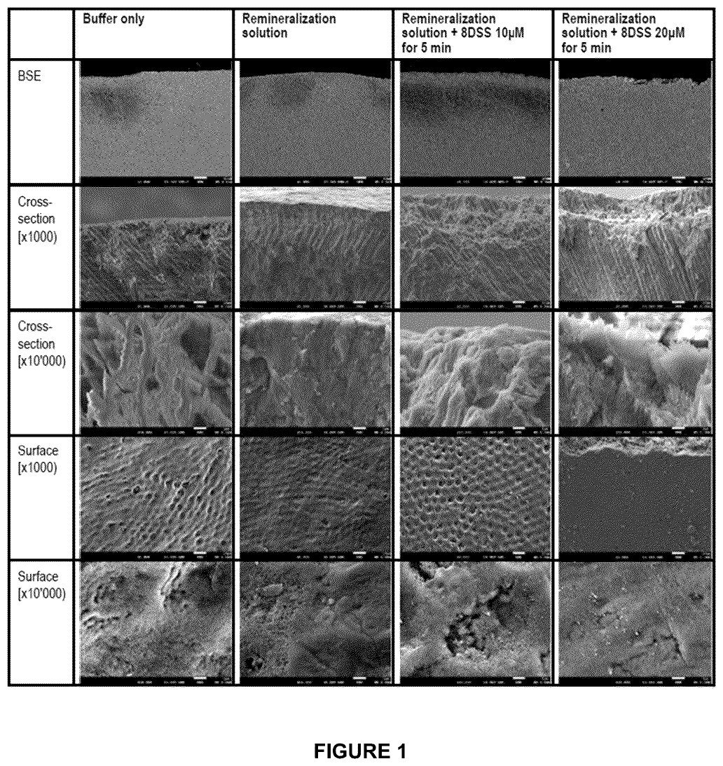

Restorative peptides, such as aspartate-serine-serine (DSS) peptides, e.g., 8DSS, facilitate dentin and enamel repairs, but are limited by their delivery. For example, as shown in FIG. 1, applying 20 μL 8DSS with a remineralization solution can enhance mineralization of the surface of caries lesions of human tooth enamel, but does not substantially affect the inside of the lesion. Briefly, extracted third molar tooth crowns were cut into quarters. Artificial caries lesions were created, and nail varnish was applied to the samples to create a 2 mm×2 mm window and reference surface. Tooth samples were sonicated for 5 minutes, rinsed, and airdried. The samples were then immersed in a solution consisting of 1.5 mM CaCl₂, 0.9 mM NaH₂PO₄, 50 mM acetate buffer, 0.1 ppm fluorine (F), pH 4.6 at 37° C. for 7 days, then rinsed under flowing distilled water for 30 seconds.

Samples were then immersed into a solution of 8DSS at a concentration of 0 μM, 10 μM or 20 M in 50 mM HEPES for 5 minutes, then exposed for 5 hours to a demineralization solution (1.5 mM CaCl, 0.9 mM NaH₂PO₄×H₂O, 50 mM acetate buffer, 0.45 ppm NaF, at pH 4.6. The samples were then rinsed, treated again for 5 minutes with the respective 8DSS solution (i.e., 0 μM, 10 μM or 20 μM 8DSS in 50 mM HEPES) and then immersed in remineralization solution (1.5 mM CaCl₂, 0.9 mM NaH₂PO₄, 20 mM HEPES, 130 mM KCl, 0.45 ppm NaF at pH to 7.0) for 19 hours. The experiment was then stopped, samples were prepared for SEM analyses by dehydration in 70 percent ethanol and cleaved through the artificial caries lesion and treatment to expose a cross section.

Samples were mounted on 45 deg SEM stubs with epoxy resin, coated with 10 nm Pt/Pd, and imaged by scanning electron microscopy (SEM) on JEOL 7900F at 5kV, current 108 μA, WD: 7.5-9.5 mm (FIG. 1). Backscattering electron (BSE) micrographs of the teeth show enhanced mineral content for teeth treated with 20 μL 8DSS for 5 minutes (FIG. 1, row 1, far right column). Likewise, SEM micrographs of the tooth surface show enhanced mineralization in teeth treated with 20 μL 8DSS for 5 minutes (FIG. 1, rows 4 and 5, far right column). SEM micrographs of the tooth interior show no clear difference between teeth treated under different conditions (FIG. 1, rows 2 and 3, columns 1-4).

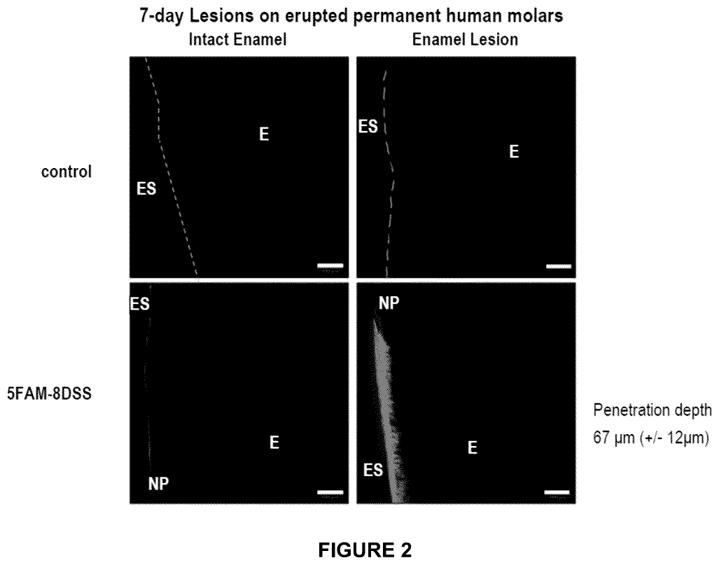

FIG. 2 shows the penetration depth of the 8DSS peptide into intact enamel and caries lesion. A standardized area was delineated on the lateral surface of human molars and an artificial caries lesion created, while in the control sample the enamel was kept intact. A solution containing 8DSS conjugated to the fluorescent tag 5FAM (5FAM-8DSS) was then applied to the delineated area and after removal of the solution and drying, the tooth was cleaved through the exposed area and analyzed by fluorescence microscopy. The images show that 5FAM-8DSS did not substantially penetrate into intact enamel and in caries lesions, the penetration was limited to approximately 67 μm (+/−12 μm) depth into the enamel from the tooth surface. These results suggest that the compositions of the present technology can be applied to the surface of a dental material or other mineralizable surface to harden the material, for example as a glass ionomer cement.

### Example 2

### Dental Self-Repair By Remineralization of Micro-Cracks Using Peptide 8DDS

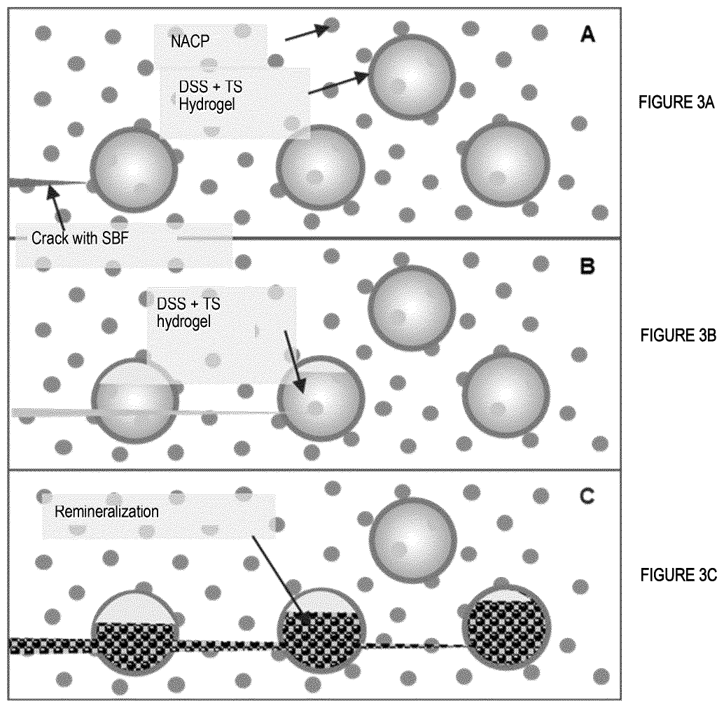

Self-healing remineralization composite materials may be created by distributing peptides throughout the composite material. As shown in FIG. 3, for example, the peptide may be distributed in hydrogels distributed throughout the composite material (e.g., FIG. 3A (DSS+TS Hydrogel)). When a crack forms, it may cross into or through the hydrogel material, causing the release of the peptide (FIG. 3B), which subsequently stimulates remineralization of the micro-crack (FIG. 3C).

Provided below is the protocol for preparing a silk-fibroin, tannic acid, 8DSS, amorphous calcium phosphate nanoparticles (NACP), and UDMA/TEGDVBE (U/V) composition for remineralization.

### Methods

### Preparation of the Silk-Fibroin Tannic Acid Hydrogel:

Step 1. Silk fibroin (SF) extraction: Silk fibroin was extracted from cocoons according to previous reports (Perry 2008). The Bombyx mori cocoons were boiled in 0.02 M sodium carbonate for 1 hour and rinsed in water to remove the sericin. The steps above were repeated three times to obtain degummed fibroin. The degummed fibroin was dried at 37° C. overnight. Then, 50 grams dry fibroin was dissolved in 200 mL 9.3 M lithium bromide aqueous solution at 60° C. for 4 hours. The regenerated silk fibroin solution was dialyzed in water and concentrated against 10% (w/v) PEG to reach a final silk fibroin concentration of 10 wt %.
Step 2. Preparation of tannic acid (TA) solution: 2 grams of tannic acid was dissolved in 20 mL 1×PBS (pH 7.4) to obtain 10 wt % tannic acid.
Step 3. Preparation of silk-fibroin tannic acid hydrogel: 700 μL of 10 wt % silk fibroin as prepared above was added with 700 μL 10 wt % tannic acid as prepared above in a centrifuge tube, vortexed for 10 seconds, then centrifuged (14.1×10³ g) for 3 minutes to obtain a silk-fibroin tannic acid hydrogel.

### Mixing the 8DSS, Amorphous Calcium Phosphate Nanoparticles (NACP), and UDMA/TEGDVBE (U/V) Mixture:

Step 1. Preparation of silk-fibroin with 40 μM 8DSS: 20 μL 8DSS (800 μM aliquot) was added to 400 μL silk-fibroin solution (10 wt %) to obtain silk-fibroin and 8DSS.
Step 2. Preparation of silk-fibroin, tannic acid, and 8DSS: 400 μL of the silk-fibroin and 8DSS solution prepared above was mixed with 400 μL tannic acid solution (10 wt % tannic acid dissolved in 1×PBS), and centrifuged to obtain a silk-fibroin, tannic acid, and 8DSS solution.
Step 3. Preparation of silk-fibroin, tannic acid, 8DSS and U/V:
   Preparation of NACP and U/V (NACP 20 wt %, U/V 20 wt %): 80 wt % U/V and 20 wt % amorphous calcium phosphate nanoparticles (NACP) was added in a 6 mL mixing cup and mixed once using a Speed Mixer at 3500 rpm for 1 minute 30 seconds.
   Preparation of silk-fibroin, tannic acid, and 8DSS and U/V (silk-fibroin, tannic acid, and 8DSS hydrogels 20 wt %, U/V 20 wt %): 20 wt % silk-fibroin, tannic acid, and 8DSS as prepared above was added in a 6 mL mixing cup. (1) 50 μL U/V was added. (2) The sample was mixed using a Speed Mixer at 3500 rpm for 1 minute 30 seconds. Steps (1) and (2) were repeated 4 times. The rest of the U/V was added to reach 80 wt %. The sample was mixed using a Speed Mixer at 3500 rpm for 1 minutes 30 seconds.
   Preparation of silk-fibroin, tannic acid, 8DSS, NACP, and U/V (Final mixture for SEM imaging and confocal laser scanning microscopy): NACP and U/V (50 wt %) and silk-fibroin, tannic acid, 8DSS, and U/V (50 wt %) were added in a 6 mL mixing cup. The sample was mixed using a Speed Mixer at 3500 rpm for 1 minute 30 seconds. The wt % of each component in silk-fibroin, tannic acid, 8DSS, NACP, and U/V was NACP 10 wt %, silk-fibroin, tannic acid, and 8DSS hydrogels 10 wt %, and U/V 80 wt %.

### Preparation of Cured Sample for SEM Imaging:

Light cure: A glass tube (2.5 mm diameter×10 mm length) was cut with a glass scriber pen. Mold release spray was applied before use. The freshly prepared silk-fibroin, tannic acid, 8DSS, NACP, and U/V mixture with camphor quinone (CQ)/amine initiator was mixed in a glass tube. A portion of the mixture was then placed on a glass slide. The sample was light cured for 3 minutes in the TRIAD 2000 curing machine to obtain a cured composite containing silk-fibroin, tannic acid, 8DSS, NACP, and U/V bar.

SEM sample prep: The cured sample was removed from the glass tube. A 1 mL centrifuge tube was obtained, and a few holes were made on its cap using a push pin needle. The sample was placed in the tube, the tube was flash frozen with liquid nitrogen, and then dried in a freeze dryer for 48 hours.

The dried sample was fractured with a razor blade and loaded on the SEM stub with the fracture surface facing up immediately after fracture. SEM glue was used to make the fractured surface parallel to the stub surface, and copper tape was applied over the top to minimize charging.

Specimens were made into 2×2×25 mm³ bars and soaked in simulated body fluid (SBF) for 5 days. The bars were fractured, and the fracture surface was imaged under SEM.

### Results

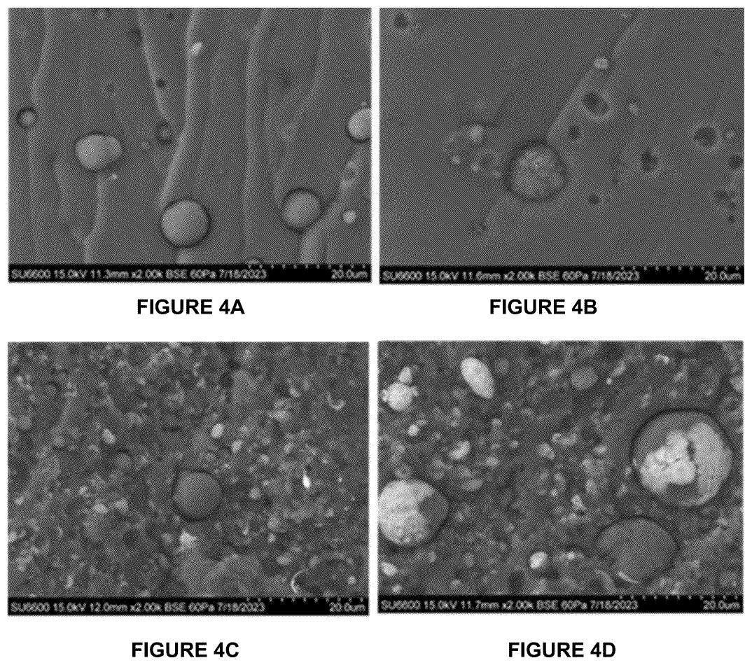

As illustrated in FIG. 4, the guided mineralization was confirmed using the combination of amorphous calcium phosphate (ACP) nanoparticles (NPs) (NACP) and a peptide, specifically 8DSS. For this experiment, four specimens were prepared to demonstrate the function of silk-fibroin, tannic acid, and U/V resin (FIG. 4A), silk-fibroin, tannic acid, and U/V resin with 8DSS peptide (FIG. 4B), silk-fibroin, tannic acid, and U/V resin with NACP (FIG. 4C), and mineralization of silk-fibroin, tannic acid, and U/V resin with 8DSS peptide+NACP (FIG. 4D). For ease of delivery, the peptide was encapsulated in micro-sized hydrogel particles (silk-fibroin hydrogel particles induced by tannic acid) that was dispersed in U/V resins.

The silk-fibroin, tannic acid hydrogel particles have a self-healing capability. They are used to disperse the 8DSS because 8DSS is water soluble while the U/V resin is not. Other hydrogel particles may also be used. The silk-fibroin, tannic acid hydrogel particles in the U/V resin (FIG. 4A) were used as a blank control. In this example, the U/V resin was an equimolar composition of urethane dimethacrylate (UDMA) and triethylene glycol divinylbenzyl ether (TEGDVBE) with camphor quinone (CQ)/amine as the photoinitiator. This mixture gave the best results in terms of handleability and mechanical performance. The results showed that new calcium phosphate minerals formed at the surface of the hydrogel in the specimen containing both NACP and the 8DSS peptide (FIG. 4D). Without the 8DSS healing agent in the hydrogel, remineralization at the hydrogel particles did not occur (FIGS. 4A-4C).

In Example 2, the silk-fibroin, tannic acid hydrogel particles were micro-sized particles. Other nano-sized hydrogel particles may be used to carry and deliver the healing agent (e.g., 8DSS) to areas that are impossible for micro-sized particles to access.

In certain embodiments, other hydrogel particles that may replace the silk-fibroin, tannic acid hydrogel particles used above include, without limitation, silk-fibroin hydrogel particles, chitosan (with/without tannic acid) hydrogel particles, alginate (with/without tannic acid) hydrogel particles, gelatin (with/without tannic acid) hydrogel particles, hyaluronic acid (with/without tannic acid) hydrogel particles, collagen (with/without tannic acid) hydrogel particles, and polyvinyl alcohol (PVA) (with/without tannic acid) hydrogel particles. Additional hydrogel particles that can be used are provided herein.

In certain embodiments, in addition to DSS peptides with various chain lengths, polyaspartic acid may also serve as the healing agent. In certain embodiments, the healing agent acts as a mineral binding agent that reacts with calcium ions and guide the formation of calcium phosphate. Additional healing agents that can be used are provided herein.

### Example 3

### Exemplary Remineralization Compositions Applied to Dentin

The silk-fibroin, tannic acid, 8DSS, NACP, and U/V composition prepared in Example 2 was further investigated and applied to dentin.

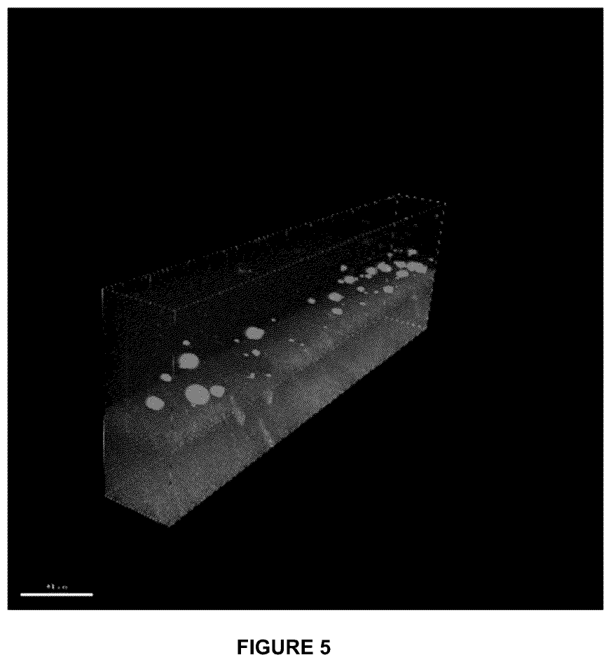

As shown in the confocal microscopy images in FIG. 5, the self-healing resin composition prepared in Example 2 was applied to dentin exposed in human tooth crowns. The silk-fibroin and tannic acid hydrogel particles (light grey spheres) with 5FAM-8DSS are shown evenly distributed adjacent to the dentin, shown in the lower portion of the image.

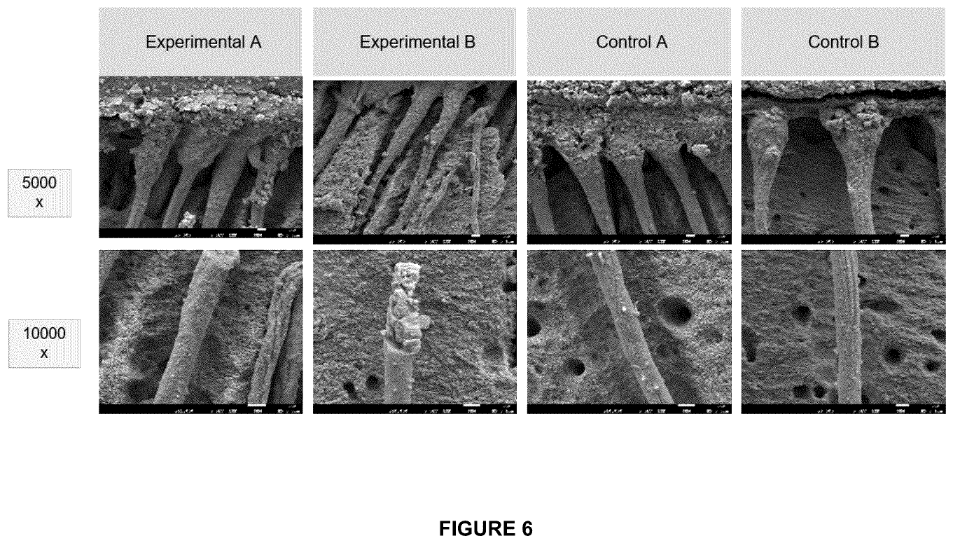

As shown in FIG. 6, when the silk-fibroin, tannic acid, 8DSS, NACP, and U/V composition prepared in Example 2 was applied to dentin, the resin infiltrated into dentin tubules and was effective in forming resin tags.

Briefly, human teeth used in all the tests were obtained according to the protocols approved by the Forsyth's Institutional Review Board. The freshly extracted teeth were treated with 0.02% (w/v) sodium azide solution and stored in deionized water at 4° C. until use. All teeth were extracted within 3 months. Teeth were embedded with Fastray composite (Harry J. Bosworth Company, Skokie, IL, USA) in cylindrical holders and ground perpendicular to their long axis with 400-grit SiC paper until the occlusal enamel was completely removed. A three-step adhesive procedure entailed: 1) etching of dentin surface with a 32% (by mass) phosphoric acid gel (Scotchbond™ Universal Etchant; 3M ESPE., Seefeld, Germany) for 15 seconds and rinsing with distilled water (after rinsing, dentin surface was kept hydrated with a moist blotting paper); 2) applying primer by brushing on the dentin surface, accumulating 5 layers, air drying between layers to evaporate the solvent; then 3) applying testing materials (i.e., by brushing once on the primed dentin surface. The entire dentin surface was then light cured for 10 seconds or 30 seconds with the use of an 8 mm tip on a quartz halogen light source having 550 mW/cm2 intensity (Spectrum 800, Caulk/Dentsply, Milford, DE, USA).

The treated tooth specimen was sectioned into 1.0 mm thick slices for resin tag evaluation. Specifically, for resin tag evaluation, the freshly cut surfaces were polished consecutively by 600-, 1200, 2400 and 4000-grit silicon carbide papers (Buehler Ltd., Lake Bluff, IL, USA). The polished surfaces were etched using 32 wt % phosphoric acid for 5 seconds and then rinsed with deionized water followed by immersion in bleach (Clorox®, Oakland, CA, USA) for 5 minutes. After deproteination, the specimens were rinsed with water. The specimens were then dehydrated by being subsequently immersed for 10minutes in ethanol solutions of ascending concentrations: 25%, 35%, 45%, 55%, 65%, 75%, 85%, 95% and 100% and vacuum dried for 24 hours. Finally, specimens were sputter-coated with gold before being observed under SEM at 15 kV.

FIG. 6 shows that the silk-fibroin, tannic acid, 8DSS, NACP, and U/V composition infiltrated into dentin tubules and was effective in forming resin tags.

### Example 4

### Preparation of Hydrogels in Different Resins

To test the ability of different resins to be used with the compositions, silk-fibroin, tannic acid (Silk-TA) hydrogels were prepared with various monomers that form resins (Table 1). The lithium phenyl-2,4,6-trimethylbenzoylphosphinate (LAP) initiator, silk-TA hydrogels, and different monomers were weighted on the same glass slide and then mixed by hand with a metal spatula. The LAP initiator was first dispersed with the monomers, and then mixed with the silk-TA hydrogels, until the samples became homogeneous. The mixture was then placed between two pieces of Mylar® Film and compressed between two glass slides, light cured for 5 minutes in a TRIAD 2000 (Dentsply). Mixtures and results are shown in Table 1.

TABLE 1
Different mixtures of resins with Silk-TA hydrogels

| | Silk-TA | | | | |
|---|---|---|---|---|---|
| Resin | hydrogels | LAP | | | After light |
| (monomers) | (g) | (g) | Resin(g) | Mixing | curing* |
| UDMA | 0.0500 | 0.0005 | 0.05 | Homogeneous, | Cured |
| | | | | good consistency | |
| | | | | and easy to | |
| | | | | handle | |
| Exothane126 | 0.0500 | 0.0005 | 0.05 | Great, | Cured, rubbery |
| | | | | Homogeneous, | and can be bent |
| | | | | good consistency | without breaking |
| | | | | and easy to | |
| | | | | handle | |
| Exothane 9 | 0.0500 | 0.0005 | 0.05 | Fare, can be | Cured |
| | | | | blended but is | |
| | | | | sticky | |
| E6 | 0.0500 | 0.0005 | 0.05 | Fare, can be | Cured |
| | | | | blended but is | |
| | | | | sticky | |
| HEMA | 0.0500 | 0.0005 | 0.05 | Poor result, the | Hard and |
| | | | | hydrogel became | transparent |
| | | | | crumbled, and | |
| | | | | HEMA is still very | |
| | | | | flowy | |
| Bis- | 0.0500 | 0.0005 | 0.05 | Blended but not | Half cured and |
| GMA/TEGDMA | | | | very | half of it is still |
| (5:5) | | | | homogeneous | soft |
| Bis- | 0.0500 | 0.0005 | 0.05 | Pretty good | Cured |
| GMA/TEGDMA | | | | | |
| (7:3) | | | | | |

*Notes on the ability of the resin to cure in the presence of the hydrogel.

The results shown in Table 1 illustrate that UDMA, Exothane 126, Exothane 9, E6, and Bis-GMA/TEGDMA (7:3) cured in the presence of the silk-fibroin, tannic acid hydrogel.

From the foregoing, it will be appreciated that specific embodiments of the disclosure have been described herein for purposes of illustration, but that various modifications may be made without deviating from the scope of the disclosure. Accordingly, the disclosure is not limited except as by the appended claims.

### REFERENCES

Perry, H, et. al. Nano-and micropatterning of optically transparent, mechanically robust, biocompatible silk fibroin films. Adv. Mater. 2008, 20, 3070-3072.

Yarborough, et al., Specific Binding and Mineralization of Calcified Surfaces by Small Peptides. Calcif Tissue Int. 2010, 86, 58-66.

1. A composition comprising:
a curable liquid;
a filler; and
an additive.

2. The composition of claim 1, wherein the curable liquid is a liquid component comprising one or more selected from the group consisting of a liquid component of a resin composite, a liquid component of a glass ionomer cement, a liquid component of a resin-modified glass ionomer cement, and a liquid component of a polyacid-modified resin composite.

3. (canceled)

4. The composition of claim 2, wherein
(a) the liquid component comprises one or more monomers in the resin composite that polymerize from a liquid state to a solid state; or
(b) the liquid component is a polymer solution of the glass ionomer cement, wherein the polymer solution is cured by a chemical reaction.

5-6. (canceled)

7. The composition of claim 2, wherein the liquid component is a mixture of monomers and polymer solutions in the resin-modified glass ionomer cement or the polyacid-modified resin composite.

8. (canceled)

9. The composition of any claim 1 , wherein the filler comprises hydrogel particles and calcium phosphate particles.

10. The composition of claim 9, wherein the hydrogel particles are made by natural materials extracted from animal products and plants, and wherein the natural materials are selected from one or more materials from the group consisting of silk fibroin (SF), tannic acid, chitosan, alginate, collagen, and gelatin; or wherein the hydrogel particles are made by synthetic materials, and wherein the synthetic materials are selected from one or more of the group consisting of hyaluronic acid, polyvinyl alcohol, polyacrylamide, poly (N-isopropylacrylamide) (PNIPAAm), poly (acrylic acid) (PAA), polyvinylpyrrolidone (PVP), poly (ethyleneimine) (PEI) polyurethane hydrogels, and poly (2-hydroxyethyl methacrylate) (PHEMA).

11. (canceled)

12. The composition of claim 9, wherein the hydrogel particles comprise about 0.1% to 30% by mass of the composition; or a size of the hydrogel particles is within the range of 20 nm to 100 μm.

13. (canceled)

14. The composition of claim 9, further comprising a healing agent.

15. The composition of claim 14, wherein the healing agent is one or more selected from the group consisting of a peptide, a bisphosphonate, and a bisphosphonate derivative.

16. The composition of claim 14, wherein the healing agent is encapsulated in the hydrogel particles or attached on the surface of the hydrogel particles; or wherein the healing agent attaches onto tooth or bone.

17. The composition of claim 14, wherein the healing agent comprises about 0.01-15% of the hydrogel particles composition by mass.

18-19. (canceled)

20. The composition of claim 15, wherein the peptide is one or more selected from the group consisting of a polyaspartic acid peptide, an aspartate-serine-serine (DSS) peptide, and an acidic peptide.

21. The composition of claim 1, further comprising a healing agent, wherein the healing agent is dispersed in curable liquid directly without hydrogel particles.

22. The composition of claim 9, wherein the composition comprises 0.1% to 30% of calcium phosphate particles; or wherein the calcium phosphate particles comprise one or more crystalline forms selected from the group consisting of amorphous calcium phosphate particles, hydroxy apatite, octacalcium phosphate, dicalcium phosphate (DCP), tricalcium phosphate (TCP), brushite, and monetite.

23. (canceled)

24. The composition of claim 9, further comprising one or more other fillers selected from the group consisting of prepolymerized fillers, silica, titanium dioxide, zirconium dioxide, fluoroaluminosilicate glass, calcium fluoride, fiber reinforcement, strontium-based glass, bioactive glass, aluminum oxide, barium oxide, yttrium oxide, magnesium oxide, calcium oxide, tin oxide, iron oxide, strontium oxide, and cerium oxide.

25. (canceled)

26. The composition of claim 1, wherein the additive comprises one or more selected from the group consisting of an initiator, a co-initiator, an accelerator, an antimicrobial agent, a pigment, an opacifier, a stabilizer, and a coupling agent.

27. The composition of claim 1, wherein the composition is one or more selected from the group consisting of a dental restorative material, a dental filling material, a laminate veneer, a dental adhesive, a denture, an adhesive, a cement, and a sealant.

28. A method of repairing a dental material comprising contacting the material with a composition comprising
a curable liquid;
a filler; and
an additive.

29. A method of treating a subject having a tooth injury comprising contacting the tooth with a composition comprising
a curable liquid;
a filler; and
an additive.

30. The composition of claim 1, wherein the composition is used for one or more selected from the group consisting of coating, high-end materials wrapping, and wound healing.
